(19) United States
(12) Patent Application Publication
(10) Pub. No.: US 2019/0099563 A1
(43) Pub. Date: Apr. 4, 2019

(54) Method and Kit for Treatment of Pulmonary Disease

(71) Applicants: Charles Joseph Shirley, Northport, AL (US); Shawn Eric Callahan, Alabaster, AL (US)

(72) Inventors: Charles Joseph Shirley, Northport, AL (US); Shawn Eric Callahan, Alabaster, AL (US)

(21) Appl. No.: 15/722,378

(22) Filed: Oct. 2, 2017

(51) Int. Cl. A61M 15/00; A61K 9/00

(52) U.S. Cl. CPC A61M 15/0046; A61K 9/0073; A61M 11/001; A61M 15/0063; A61M 15/003

(57) ABSTRACT

The present embodiment relates to the use of liquid terbutaline sulfate from a nebulizer. Liquid terbutaline sulfate is not typically available in plastic dosage vials. Rather, liquid terbutaline sulphate is most commonly packaged in capped glass ampules such as those used for injectable drugs. In view of the effectiveness of liquid terbutaline sulphate in treating pulmonary and bronchial constriction diseases, a method and apparatus that can be employed by pharmacists and patients allowing for the increased ease of use of liquid terbutaline sulphate from capped glass ampules in nebulizers is disclosed.

### CROSS-REFERENCE TO RELATED APPLICATIONS

Not Applicable.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISK APPENDIX

Not Applicable

### FIELD OF THE INVENTION

The present invention relates to method and system for improving patient use of a bronchodilator to treat pulmonary and bronchial constriction diseases arising from both short and long term illness.

### BACKGROUND OF THE INVENTION

Pulmonary and bronchial constriction diseases such as Asthma, Chronic Obstructive Pulmonary Disease (COPD) and others are routinely treated by administering an aqueous solution containing a bronchodilator drug in aerosolized form from a nebulizer. Common bronchodilators employed to treat such diseases in aerosolized form from a nebulizer are, among others, albuterol, levalbuterol, and ipratropium bromide. Such drugs are typically prescribed and delivered to the patient in small plastic vials containing a pre-measured liquid dosage solution. In order to access the medicine in such vials, the patient must tear off a crimped top section of the vial so that the patient can pour the aqueous solution into the designated chamber on a nebulizer. Patients with physical limitations or disabilities, such as elderly patients, often find it difficult to administer their treatments without assistance because their physical limitations make it virtually impossible to open such vials. Another drug that is effective in the treatment of pulmonary and bronchial constriction diseases is liquid terbutaline sulfate. Although liquid terbutaline sulfate is also effective when used from a nebulizer, such use is atypical because liquid terbutaline sulfate is not usually available in plastic dosage vials. Rather, liquid terbutaline sulphate is most commonly packaged in capped glass ampules such as those used for injectable drugs. In view of the effectiveness of liquid terbutaline sulphate in treating pulmonary and bronchial constriction diseases, a method and apparatus that can be employed by pharmacists and patients allowing for the increased ease of use of liquid terbutaline sulphate from capped glass ampules in nebulizers is desirable. Such apparatus and methods for use of liquid terbutaline sulphate stored in capped ampules are contemplated herein.

### SUMMARY OF THE INVENTION

The primary object of the present invention is to provide apparatus and methods for the use by pharmacists and patients of liquid terbutaline sulphate packaged in injectable capped glass ampules in the treatment of pulmonary and bronchial constriction diseases from a nebulizer. Accordingly, the present invention, in one embodiment, contemplates a process in accordance with the present invention that may include the steps of
1. A pharmacy filling a prescription for liquid terbutaline sulphate packaged in capped glass ampules;
2. A pharmacy training the patient to de-cap a capped glass ampule of liquid terbutaline sulphate;
3. A pharmacy training the patient to extract the liquid terbutaline sulfate from a capped glass ampule;
4. A pharmacy training the patient to mix the extracted liquid terbutaline sulphate with a prescribed amount of another respiratory drug or sodium chloride in the nebulizer.
5. A pharmacy providing training to the patient in proper use of the patient's nebulizer.

In another embodiment, there is contemplated a process in accordance with the present invention that may include the steps of
1. A pharmacy filling a prescription for liquid terbutaline sulphate packaged in capped glass ampules;
2. A pharmacy providing the patient with a kit comprising a de-capper and a base having a receiving chamber for receiving and holding a capped glass ampule of liquid terbutaline sulphate;
3. A pharmacy training the patient to use the kit items as necessitated by the patient's needs to de-cap a capped glass ampule of liquid terbutaline sulphate;
4. A pharmacy training the patient to extract the liquid terbutaline sulfate from a capped glass ampule;
5. A pharmacy training a patient to mix the extracted liquid terbutaline sulphate with a prescribed amount of another respiratory drug or sodium chloride in the nebulizer.
6. A pharmacy providing training to the patient in proper use of the patient's nebulizer.

The above described and other features and advantages of the present disclosure will be appreciated and understood by those skilled in the art from the following detailed description, drawings and appended claims.

### BRIEF DESCRIPTION OF THE DRAWINGS

The object and features of the present invention will become more fully apparent from the following description, taken in conjunction with the accompanying drawings. The following figures depict presently preferred embodiment of the present invention and are, therefore, not to be considered limiting of its scope.

FIG. 1 is a flow chart depicting an exemplary embodiment of the present invention;

FIG. 2 is a flow chart depicting a second exemplary embodiment of the present invention;

### DETAILED DESCRIPTION OF THE INVENTION

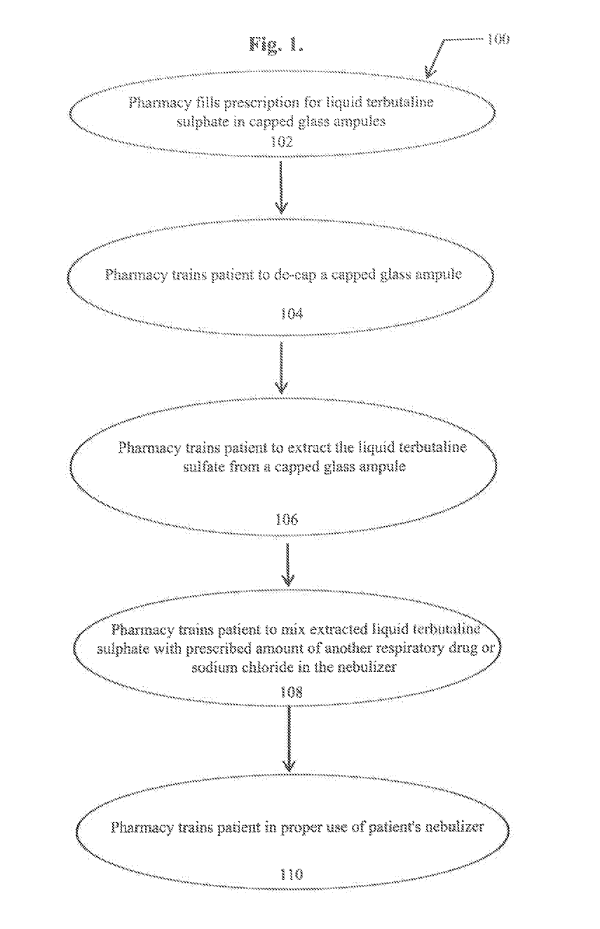

Referring now to the invention in more detail, in FIG. 1 there is shown an exemplary process of a pharmacy providing apparatus and methods for the use by patients of terbutaline sulphate packaged in injectable capped glass ampules in the treatment of pulmonary and bronchial constriction diseases from a nebulizer 100. The method 100 may include a pharmacy filling a patient's prescription for liquid terbutaline sulphate in capped glass ampules 102; A pharmacy training the patient to de-cap a capped glass ampule of liquid terbutaline sulphate 104; A pharmacy training the patient to extract the liquid terbutaline sulfate from a capped glass ampule 106; A pharmacy training the patient to mix the extracted liquid terbutaline sulphate with a prescribed amount of another respiratory drug or sodium chloride in the nebulizer 108; A pharmacy providing training to the patient in proper use of the patient's nebulizer 110;

The method 100 may comprise filling a patient's prescription with an approved derivative of liquid terbutaline sulphate in capped glass ampules 102.

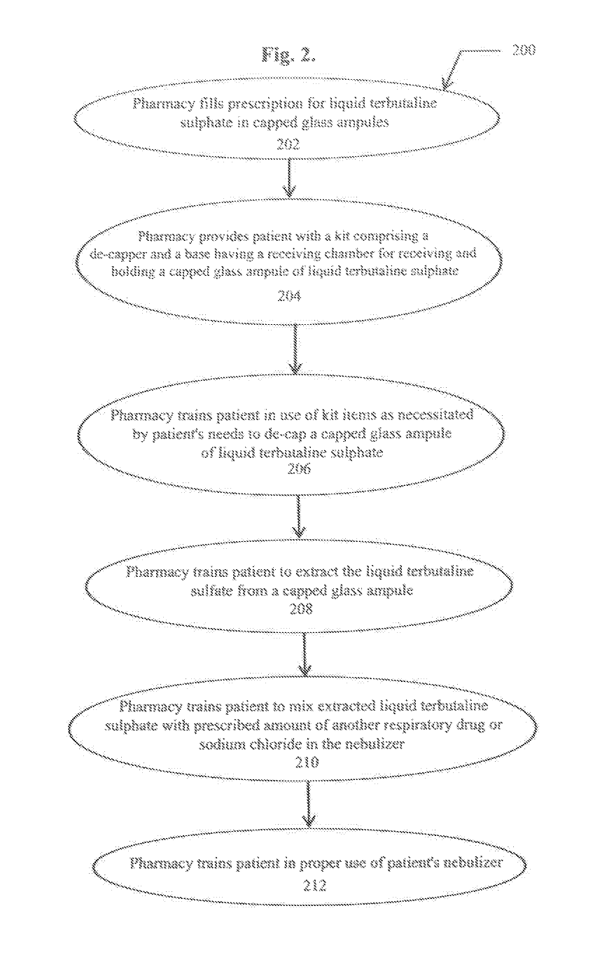

Referring now to FIG. 2 there is shown an exemplary process of a pharmacy providing apparatus and methods for the use by patients of terbutaline sulphate packaged in injectable capped glass ampules in the treatment of pulmonary and bronchial constriction diseases from a nebulizer 200. The method 200 may include a pharmacy filling a patient's prescription for liquid terbutaline sulphate in capped glass ampules 202; A pharmacy providing the patient with a kit comprising a de-capper and a base having a receiving chamber for receiving and holding a capped glass ampule of liquid terbutaline sulphate 204; A pharmacy training the patient to use the kit items as necessitated by the patient's needs to de-cap a capped glass ampule of liquid terbutaline sulphate 206; A pharmacy training the patient to extract the liquid terbutaline sulfate from a capped glass ampule 208; A pharmacy training a patient to mix the extracted liquid terbutaline sulphate with a prescribed amount of another respiratory drug or sodium chloride in the nebulizer 210; A pharmacy providing training to the patient in proper use of the patient's nebulizer 212.

The method 200 may include the pharmacy selecting one of several different types of approved de-capping devices for inclusion in the kit 204 based on the individual patient's needs, physical limitations and dexterity.

The method 200 may include the pharmacy selecting a base having a receiving chamber for receiving and holding a capped glass ampule in varying shapes and sizes for inclusion in the kit 204 based on the individual patient's needs, physical limitations and dexterity.

While the foregoing written description of the exemplary embodiments of the invention enables one of ordinary skill to use what is considered presently to be the best method thereof, it has been provided for the purposes of illustration and description. It is not intended to be exhaustive or to limit the invention to the precise form disclosed. Combinations of the above arrangements, and other arrangements not specifically described herein, will be apparent to those of skill in the art upon reviewing the above description. Those of ordinary skill will understand and appreciate the existence of variations, combinations, and equivalents of the specific embodiment and examples herein. The invention should therefore not be limited by the above described embodiment, method, and examples, but by all embodiments and methods within the scope and spirit of the invention as claimed.

What is claimed is:

1. A method using liquid terbutaline sulphate from capped glass ampules to treat pulmonary and bronchial constriction diseases from a nebulizer, the method comprising:
a pharmacy filling a patient's prescription for liquid terbutaline sulphate in capped glass ampules;
a pharmacy training the patient to de-cap a capped glass ampule of liquid terbutaline sulphate;
a pharmacy training the patient to extract the liquid terbutaline sulfate from the capped glass ampule;
a pharmacy training the patient to mix the extracted liquid terbutaline sulfate with a prescribed amount of another respiratory drug or sodium chloride in the nebulizer;
a pharmacy providing training to the patient in proper use of the patient's nebulizer.

2. A method using liquid terbutaline sulphate from capped glass ampules to treat pulmonary and bronchial constriction diseases from a nebulizer, the method comprising:
a pharmacy filling a prescription for liquid terbutaline sulphate packaged in capped glass ampules;
a pharmacy providing the patient with a kit comprising a de-capper and a base having a receiving chamber for receiving and holding a capped glass ampule of liquid terbutaline sulphate;
a pharmacy training the patient to use the kit items as necessitated by the patient's needs to de-cap a capped glass ampule of liquid terbutaline sulphate;
a pharmacy training the patient to extract the liquid terbutaline sulfate from the capped glass ampule;
a pharmacy training the patient to mix the extracted liquid terbutaline sulfate with a prescribed amount of another respiratory drug or sodium chloride in the nebulizer;
a pharmacy providing training to the patient in proper use of the patient's nebulizer.

3. A kit for de-capping a capped glass ampule of liquid terbutaline sulphate comprising:
a de-capping device; and
a base having a receiving chamber for receiving and holding a capped glass ampule of liquid terbutaline sulphate.
